(19) United States
(12) Patent Application Publication
(10) Pub. No.: US 2025/0002785 A1
(43) Pub. Date: Jan. 2, 2025

(54) MULTIPURPOSE IMIDAZOLINE-BASED PROCESS CHEMICALS

(71) Applicant: Baker Hughes Oilfield Operations LLC, Houston, TX (US)

(72) Inventors: Enrico T. Nadres, Houston, TX (US); Joseph Arensdorf, Conroe, TX (US); Frances H. De Benedictis, Houston, TX (US); Matthew Barnes, Conroe, TX (US); Analette Carrasco, Missouri City, TX (US); Bradley G. Harrell, Pearland, TX (US); Onome Ugono, Rosenberg, TX (US)

(73) Assignee: Baker Hughes Oilfield Operations LLC, Houston, TX (US)

(21) Appl. No.: 18/213,776

(22) Filed: Jun. 23, 2023

(51) Int. Cl. C09K 15/30; C07D 233/16; C09K 8/54

(52) U.S. Cl. CPC C09K 15/30; C07D 233/16; C09K 8/54; C09K 2208/20

(57) ABSTRACT

Multipurpose process chemicals have been developed, together with methods for producing and using the multipurpose process chemicals. In one aspect, a method for using a multipurpose process chemical includes the steps of reacting an aminoethyl imidazoline with a cyclic carbonate to form the multipurpose process chemical, and injecting the multipurpose process chemical into a system to address at least one of metal corrosion or hydrogen sulfide accumulation. In another aspect, a method for preparing a multipurpose process chemical includes the steps of obtaining an imidazoline synthesized from a fatty acid; combining the imidazoline with a cyclic carbonate to form a mixture; and heating the mixture at approximately 80° C. to synthesize the at least one adduct from the imidazoline and the cyclic carbonate.

### FIELD OF THE INVENTION

The present application is generally directed at chemical formulations, and in particular to multipurpose process chemicals useful for corrosion inhibition and hydrogen sulfide scavenging, and methods for preparing such process chemicals.

### BACKGROUND

The success of many industrial processes depends on the creation and protection of reliable infrastructure. The oil and gas industry, for example, has continued to improve the equipment available for the exploration, production, transport, storage, refining, and distribution of oil and gas resources. There is an ongoing need to maintain this equipment and to protect existing infrastructure from corrosion, hydrogen sulfide fouling, and other conditions that may cause mechanical failure.

Various corrosion inhibitors have been developed to combat corrosion in various types of metal equipment. Among these, imidazolines are one of the most widely used and effective corrosion inhibitors. Certain imidazolines may be derived from a reaction of esters or fatty acids (e.g., tall oil and oleic acid) with polyethylene amines, such as diethylenetriamine (DETA), triethylenetetramine (TETA), tetraethylenepentamine (TEPA), and (2-aminoethyl)ethanolamine (AEEA). The ratio of ester or fatty acid to polyethylene amine used to synthesize the imidazolines is typically 1:1. In general, imidazolines exhibit low toxicity and good biodegradability in the environment. Imidazoline-based corrosion inhibitors are also soluble in hydrocarbon or water-alcohol-based solutions; however, these corrosion inhibitors are generally limited in their applicability because they are not readily dispersible in water.

Several methods have been employed to enable the dispersion of imidazoline-based corrosion inhibitors in water. These methods include quaternization, acidification (i.e., dissolving in acids), and oxyalkylation of the imidazoline compounds. However, each of these methods has significant drawbacks in application. For example, quaternization and acidification are typically accomplished using chloride and acetate salts, both of which can promote corrosion in metal equipment. Oxyalkylation procedures usually involve an extra procedural step of transferring the product to a specialized plant, making the procedure difficult to implement in existing infrastructure.

In addition to introducing corrosion inhibitors into equipment, such as those used in the oil and gas industry, operators often introduce separate chemicals to combat issues such as hydrogen sulfide fouling. The uncontrolled presence of hydrogen sulfide, which occurs naturally in crude oil, creates operational challenges related to corrosion, safety, and odor. Hydrogen sulfide scavengers such as hexahydro-1,3,5-tri-(hydroxyethyl)-s-triazine (commonly referred to as MEA triazine) and N, N′-methylenebis-oxazolines are commonly added to equipment to address the presence of hydrogen sulfide. The introduction of multiple classes of chemicals to address different issues of equipment maintenance creates additional expense and logistical burden.

The high cost of equipment maintenance across industries has created market demand for more effective chemistries to address issues such as corrosion and hydrogen sulfide accumulation. It is desirable for these chemistries to be easily incorporated into existing processes. Further, the increased emphasis on addressing environmental concerns is creating widespread industrial impetus to seek chemistries that are more environmentally friendly. There is, therefore, a need for cost-effective and environmentally friendly treatments for use in equipment maintenance in various industries. The present disclosure is directed at these and other deficiencies in the prior art.

### SUMMARY OF THE INVENTION

The inventive concepts disclosed herein are generally directed to multipurpose process chemicals and methods for preparing such multipurpose process chemicals. In one aspect, a method for using a multipurpose process chemical includes the steps of reacting an aminoethyl imidazoline with a cyclic carbonate to form the multipurpose process chemical, and injecting the multipurpose process chemical into a system to address at least one of metal corrosion or hydrogen sulfide accumulation.

In another aspect, a multipurpose process chemical includes a first adduct (i) of the structure:

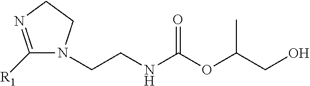

where R₁ is a C₈-C₂₂ alkenyl; and a second adduct (ii) of the structure:

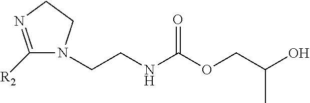

where R₂ is a C₈-C₂₂ alkenyl.

In another aspect, a method is disclosed for preparing a multipurpose process chemical that includes at least one adduct. In these embodiments, the method includes the steps of obtaining an imidazoline synthesized from a fatty acid; combining the imidazoline with a cyclic carbonate to form a mixture; and heating the mixture at approximately 80° C. to synthesize the at least one adduct from the imidazoline and the cyclic carbonate.

### BRIEF DESCRIPTION OF THE DRAWINGS

FIG. 1 illustrates an NMR graph for various imidazolines.

FIG. 2 illustrates an IR graph for the imidazolines of FIG. 1.

FIG. 3 illustrates an exemplary reaction of imidazoline with a cyclic carbonate to synthesize a multipurpose process chemical that includes a mixture of adducts.

FIG. 4 depicts the NMR results for the imidazoline and the reaction product of FIG. 3.

FIG. 5 illustrates an NMR graph comparing the signals for propylene carbonate, oleyl imidazoline, and the reaction product of various imidazolines with propylene carbonate.

FIG. 6 depicts an exemplary reaction of imidazoline with propylene carbonate.

FIG. 7 depicts an exemplary reaction of imidazoline with propylene carbonate in which excess propylene carbonate is present in the resulting multipurpose process chemical.

FIG. 8 illustrates the dispersibility of an exemplary embodiment of the multipurpose process chemical in water and aromatic solvent compared to its parent imidazoline.

### DETAILED DESCRIPTION

It has been discovered that a chemical formulation can be synthesized and used as a multipurpose process chemical for inhibiting metal corrosion and hydrogen sulfide accumulation. As used herein, the term “adduct” refers to a reaction product of two or more distinct molecules, where the reaction product contains all atoms of the two or more distinct molecules.

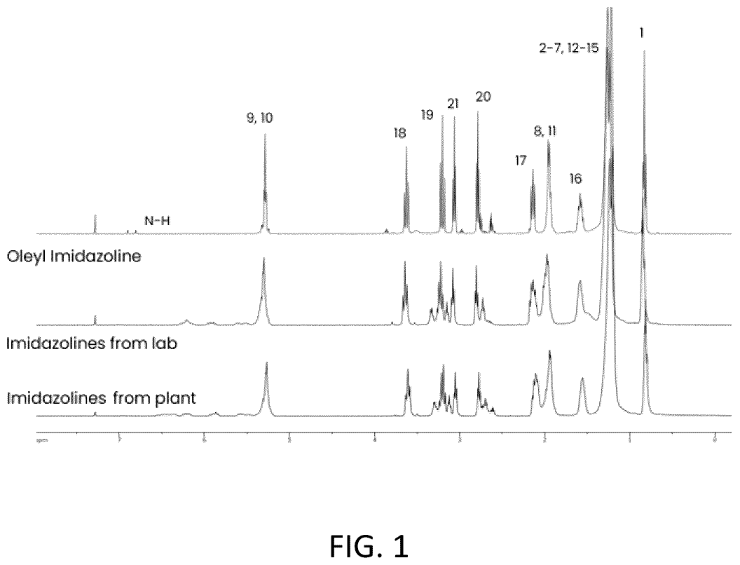

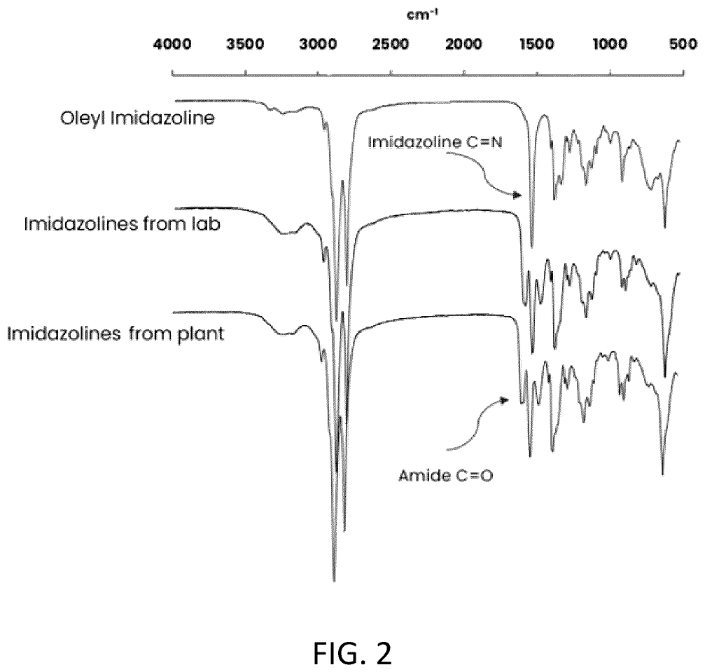

In one embodiment, a method for using a multipurpose process chemical includes the steps of reacting an imidazoline with a cyclic carbonate to form the multipurpose process chemical and injecting the multipurpose process chemical into a system to address at least one or metal corrosion or hydrogen sulfide accumulation. The imidazoline may be an aminoethyl imidazoline that is derived from a condensation reaction of a fatty acid and a polyethylene amine. The imidazoline resulting from the condensation reaction will have a practical concentration of 0.5 eq. of reactive imidazoline (with 1:1 fatty acid to polyethylene amine) and 0.5 eq. of non-reactive imidazoline (with 2:1 fatty acid to polyethylene amine). Suitable fatty acids include but are not limited to tall oil fatty acid and oleic acid. In some embodiments, the polyethylene amine may be diethylenetriamine (DETA). In other embodiments, the polyethylene amine may be triethylenetetramine (TETA), tetraethylenepentamine (TEPA), or (2-aminoethyl)ethanolamine (AEEA). FIG. 1 illustrates an NMR graph comparing the signals for various imidazolines synthesized from fatty acid and polyethylene amine. FIG. 2 provides an IR graph for the same.

Suitable cyclic carbonates for reaction with the imidazoline include but are not necessarily limited to propylene carbonate, ethylene carbonate, glycerol carbonate, vinyl carbonate, and combinations thereof.

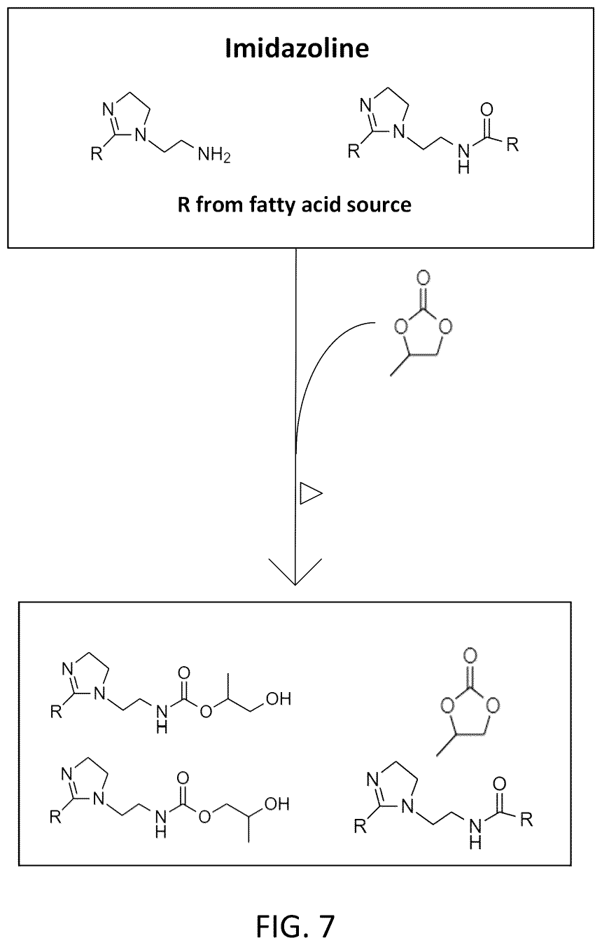

The ratio of the imidazoline and the cyclic carbonate (imidazoline:cyclic carbonate) used in the reaction may be in a range from about 1:0.5 to about 1:2, including the discrete ratios of approximately 1:0.5, 1:1, 1:1.5, or 1:2. In some embodiments, the amount of cyclic carbonate in the reaction exceeds what is required to react all of the imidazoline, thereby leaving excess unreacted cyclic carbonate in the multipurpose process chemical when the reaction is complete. FIG. 7 depicts an exemplary embodiment in which the reaction of imidazoline with propylene carbonate results in excess unreacted propylene carbonate when the reaction is complete.

In some embodiments, the reaction of the imidazoline and the cyclic carbonate may be conducted at a temperature between about 50° C. and about 120° C. For some embodiments, the reaction temperature is approximately 80° C.

For applications in the oil and gas industry, the multipurpose process chemical may be injected into oil and gas wells or equipment to address one or more concerns, including but not limited to, metal corrosion and hydrogen sulfide fouling. The method of using the multipurpose process chemical may include the step of mixing the multipurpose process chemical into a target fluid, where the target fluid contains hydrogen sulfide. The multipurpose process chemical of the presently disclosed embodiments may be injected in a concentration between about 1 ppm to about 800 ppm of the multipurpose process chemical in the target fluid. In some embodiments, the multipurpose process chemical is injected in an amount of about 400 ppm of the multipurpose process chemical in the target fluid.

Although applications for the multipurpose process chemical in the oil and gas industry are specifically contemplated above, it will be appreciated that the multipurpose process chemicals may be applied in any aqueous system that is susceptible to metal corrosion and/or hydrogen sulfide accumulation. For example, the multipurpose process chemicals may be used for treatment in aqueous systems including, but not limited to, cooling water systems, petroleum production, oil recovery (well casing, pipelines), refining, geothermal wells, and other oil field applications; boiler and boiler water systems, power generation and mineral process water including washing, flotations; paper mill digesters, washers, bleach plants, white water systems, mill water systems, black liquor evaporators in the pulp and paper industry, gas scrubbers and air washers; continuous casting processes in the metallurgical industry; air conditioning and refrigeration systems; building fire protection heating water, such as pasteurization water; water reclamation and purification systems; membrane filtration water systems; food processing streams and waste treatment systems as well as in clarifiers, liquid-solid applications, municipal sewage treatment systems; and industrial or municipal water distribution systems.

In another embodiment, the multipurpose process chemical includes at least one adduct. The structure(s) of the at least one adduct in the multipurpose process chemical will vary depending on the structures of the imidazoline and the cyclic carbonate used to synthesize the at least one adduct. In a non-limiting embodiment, the multipurpose process chemical may include an adduct (i) that is prepared by a reaction of the imidazoline and propylene carbonate, where the adduct (i) has the following structure:

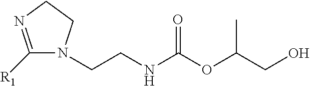

where R₁ is a C₈-C₂₂ alkenyl. In another non-limiting embodiment, the multipurpose process chemical may include an adduct (ii) prepared by a reaction of the imidazoline and propylene carbonate, where the adduct (ii) has the structure:

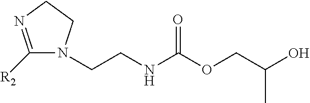

where R₂ is a C₈-C₂₂ alkenyl. In some embodiments, the multipurpose process chemical may include a mixture of both foregoing adducts (i), (ii). In the process chemical of such embodiments, the foregoing adducts (i), (ii) may be present in similar quantities, or one adduct may be present in the process chemical in a greater quantity than the other adduct.

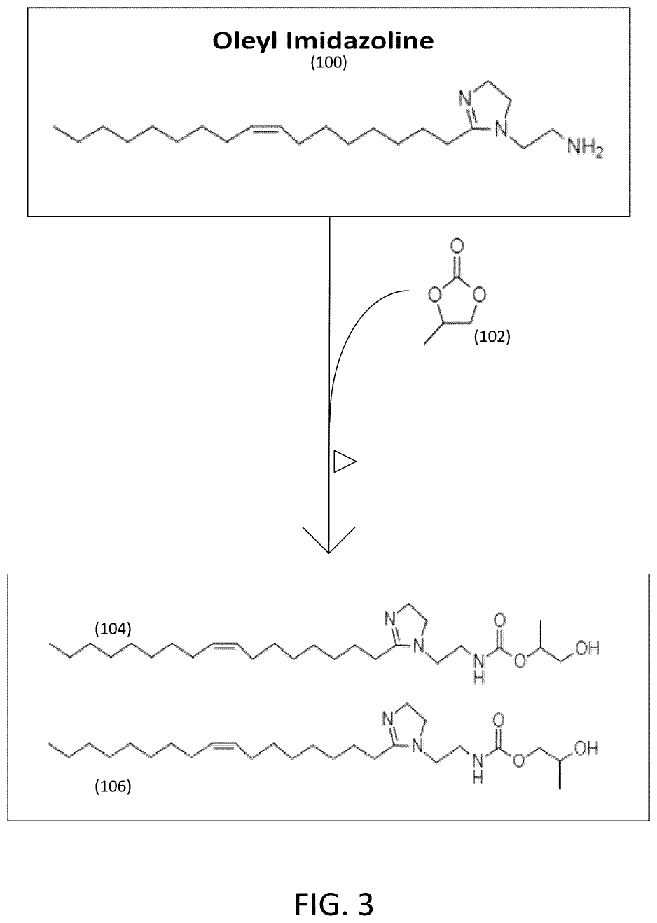

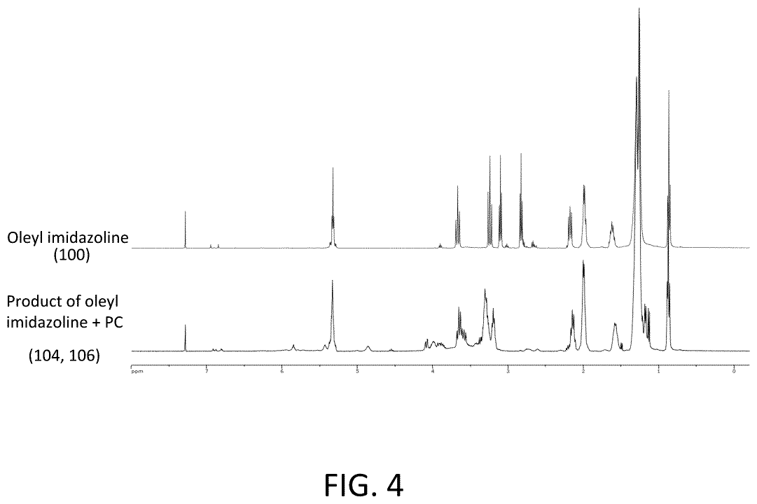

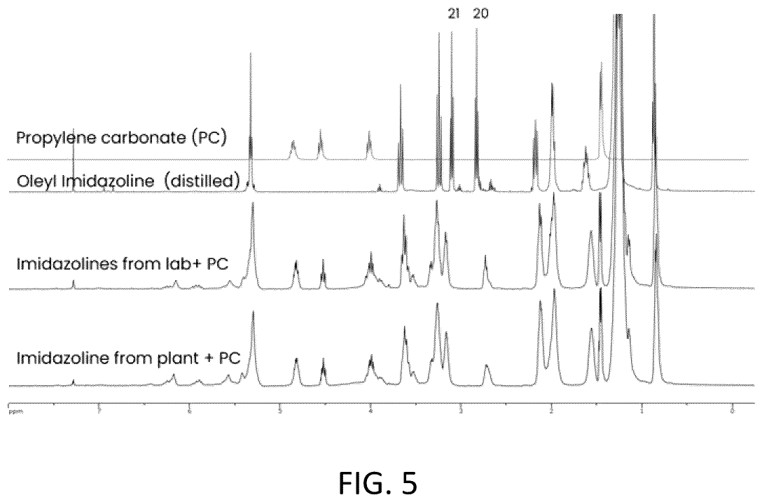

FIG. 3 illustrates an exemplary reaction of an oleyl imidazoline 100 with propylene carbonate 102 to form a mixture of adducts 104, 106. FIG. 4 depicts a resulting NMR graph with signals for the oleyl imidazoline 100 and the adducts 104, 106 for this exemplary reaction. FIG. 5 further depicts an NMR graph that compares propylene carbonate, oleyl imidazoline, and the reaction product of various other imidazolines with propylene carbonate.

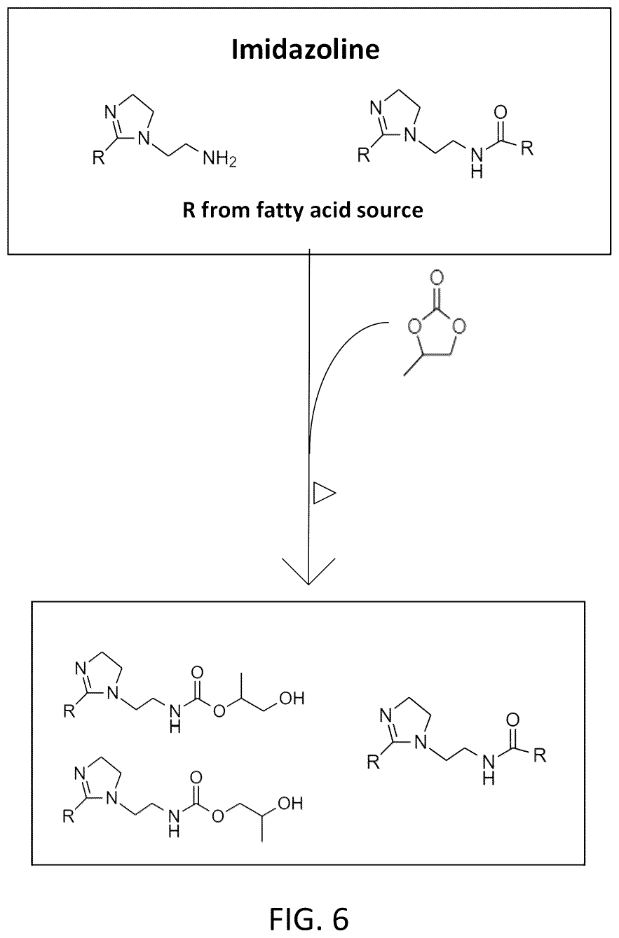

In some cases, the multipurpose process chemical further includes a non-reactive compound of the structure:

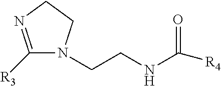

where R₃ is a C₈-C₂₂ alkenyl, and R₄ is an alkyl group. Unlike the foregoing adducts (i) and (ii), this compound is not synthesized by the reaction of the imidazoline and propylene carbonate. Rather, this compound may be synthesized alongside the imidazoline during the initial condensation reaction of fatty acid and polyethylene amine. The R₄ alkyl group is derived from the fatty acid of this condensation reaction. FIGS. 6 and 7 illustrate exemplary embodiments in which the non-reactive compound is present with the active imidazoline before the reaction and in the multipurpose process chemical after the addition of propylene carbonate.

In general, imidazolines are readily dispersible in aromatic solvents. The multipurpose process chemicals retain comparable dispersibility properties to its parent imidazoline. That is, the process chemical is also readily dispersible in aromatic solvent. Imidazolines, however, generally do not disperse readily in water. Unlike its parent imidazoline, in this case, the process chemical demonstrates good dispersibility in water. Because the multipurpose process chemical exhibits good dispersibility in both aromatic solvent and water, a wider range of compatibility is possible during formulation.

In another embodiment, a method is disclosed for preparing the multipurpose process chemicals, where the multipurpose process chemicals include at least one adduct. The method includes the steps of obtaining imidazoline synthesized from a fatty acid; contacting the imidazoline with cyclic carbonate to form a mixture; and heating the mixture at approximately 80° C. to synthesize the at least one adduct from the imidazoline and the cyclic carbonate. In some embodiments, the step of obtaining an imidazoline involves deriving the imidazoline from a condensation reaction of tall oil fatty acid and diethylenetriamine (DETA).

In some embodiments, the method further includes the step of heating the imidazoline to approximately 80° C. before contacting the imidazoline with the cyclic carbonate to form the mixture. Inclusion of this step is preferable for scaled up production. In such production, the cyclic carbonate may be contacted with the imidazoline by stepwise or dropwise addition. It is anticipated that this method of preparing the multipurpose process chemical can be easily incorporated into existing manufacturing infrastructure, without the need to transfer the product to a specialized plant.

### EXAMPLE I

Tests were performed to compare the corrosion inhibition provided by the multipurpose process chemicals prepared from distilled imidazolines. The following samples were obtained:

| | |
|---|---|
| Sample I-A | The reaction products from the condensation |
| | of oleic acid and diethylene triamine |
| Sample I-B | Distilled imidazoline |
| Sample I-C | Multipurpose process chemical from the reaction |
| | of distilled imidazoline with propylene carbonate |
| Sample I-D | Pure propylene carbonate |

Sample I-B was prepared by isolating the major component of Sample I-A (imidazoline) by vacuum distillation. Sample I-C was prepared by mixing 165.6 grams (˜0.5 mol) of distilled imidazoline with 51.7 grams (0.5 mol) of propylene carbonate (i.e., approximately 1:1 distilled imidazoline to propylene carbonate) in a three-neck flask. A nitrogen gas blanket was introduced. The mixture was then stirred at 300 rpm, heated to 80° C. for one (1) hour, and subsequently cooled. The resulting multipurpose process chemical contained excess propylene carbonate.

Samples I-A through I-D were evaluated for corrosion inhibition performance at a concentration of 10 ppm for the samples. As demonstrated in the below table, Sample I-A demonstrated a modest inhibition of fifty percent (50%). For Sample I-B, the activity increased to sixty-two percent (62%). Further increase in activity was achieved by the reaction of the distilled imidazoline with propylene carbonate, bringing the inhibition of Sample I-C to ninety-one percent (91%). This increase in performance is attributed to the formation one or more adducts from the reaction. Sample I-D demonstrated no corrosion inhibiting activity.

| Sample | % Inhibition @ 10 ppm Active |
|---|---|
| I-A | 50 |
| I-B | 62 |
| I-C | 91 |
| I-D | 0 |

### EXAMPLE II

Tests were also performed to compare the corrosion inhibition performance of a tall oil imidazoline with the multipurpose process chemical prepared by the reaction of the same tall oil imidazoline and propylene carbonate. Sample II-A was obtained from the condensation reaction of tall oil and diethylene triamine. Sample II-B was prepared by mixing 165.6 grams (˜0.5 mol) of Sample II-A with 25.5 grams (0.25 mol) of propylene carbonate (i.e., approximately 1:0.5 tall oil imidazoline to propylene carbonate) in a three-neck flask. Sample II-C was similarly prepared by mixing 165.6 grams (˜0.5 mol) of Sample II-A with 51.7 grams (0.5 mol) of propylene carbonate (i.e., approximately 1:1 tall oil imidazoline to propylene carbonate) in a three-neck flask. A nitrogen gas blanket was introduced to the flasks for both Samples II-B and II-C, and the mixtures were stirred at 300 rpm, heated to 80° C. for one (1) hour, and subsequently cooled. Sample II-B contained no excess propylene carbonate. In contrast, the multipurpose process chemical of Sample II-C contained excess propylene carbonate.

Each of the samples were tested for corrosion inhibition performance at a concentration of 10 ppm for each of the samples. As demonstrated in the below sample, Sample II-B demonstrated lower corrosion inhibition (64.5%) than Sample II-A (89.4%). However, when the tall oil imidazoline and propylene carbonate were reacted in equimolar amount for Sample II-C, the activity was better than that of Sample II-A.

| Sample | % Inhibition @ 10 ppm Active |
|---|---|
| II-A | 89.4 |
| II-B | 64.5 |
| II-C | 95.3 |

### EXAMPLE III

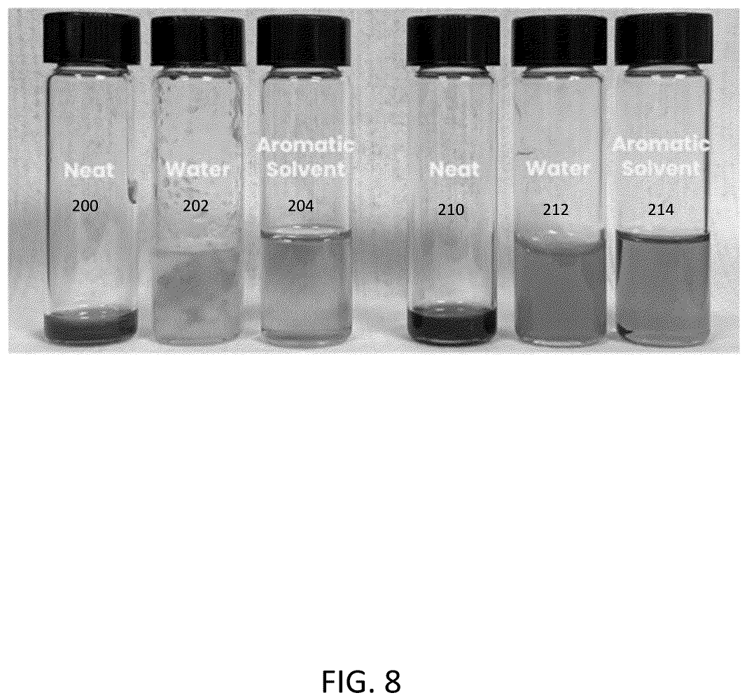

An additional test was performed to compare water dispersibility of an exemplary embodiment of the multipurpose process chemicals with the parent imidazoline. The multipurpose process chemical was obtained by mixing propylene carbonate with the parent imidazoline, then heating the mixture at 130° C. until all imidazoline was converted into one or more adducts. As depicted in FIG. 8, a neat sample of the parent imidazoline was placed in first vial 200, the parent imidazoline was placed with water in a second vial 202, and the parent imidazoline was placed with an aromatic solvent in a third vial 204. FIG. 8 further depicts a fourth vial 210 containing a neat sample of the multipurpose process chemical, a fifth vial 212 containing the process chemical with water, and a sixth vial 214 containing the process chemical with the aromatic solvent. Visual inspection revealed that the multipurpose process chemical exhibited improved dispersibility in water and similar dispersibility in aromatic solvent when compared to the parent imidazoline.

### EXAMPLE IV

An additional test was performed to compare the corrosion inhibition performance of Sample II-C (the multipurpose process chemical derived from the tall oil imidazoline of Sample II-A and propylene carbonate in a 1:1 ratio) with water-dispersible corrosion inhibitors obtained from Sample II-A by known methods. Sample IV-A was obtained by oxyalkylation of Sample II-A. Sample IV-B was obtained by the quaternization of Sample II-A. Sample IV-C was obtained by acidizing Sample II-A with acetate salt. As demonstrated in the table below, the corrosion inhibiting activity of Sample II-C was higher than that of the known water-dispersible corrosion inhibitors (Samples IV-A, IV-B, and IV-C).

| Sample | % Inhibition @ 10 ppm Active |
|---|---|
| II-A | 89.4 |
| II-C | 95.3 |
| IV-A | 73.1 |
| IV-B | 63.1 |
| IV-C | 88.3 |

### EXAMPLE V

Further testing was performed to compare the hydrogen sulfide scavenging properties of an exemplary embodiment of the multipurpose process chemical compared to conventional scavengers.

A sour Isopar™ M solvent was obtained with 4000 ppm hydrogen sulfide (H₂S). Samples were obtained by mixing the following with the sour Isopar™ M solvent:

| Control | Nothing |
|---|---|
| Sample V-A | 400 ppm of Baker Hughes' SULFIX ™ 9272 |
| | H₂S scavenger |
| Sample V-B | 400 ppm of Baker Hughes' SULFIX ™ 9292 |
| | H₂S scavenger |
| Sample V-C | 800 ppm of propylene carbonate in |
| | isopropyl alcohol |
| Sample V-D | 1600 ppm of propylene carbonate in |
| | isopropyl alcohol |
| Sample V-E | 400 ppm of the multipurpose process chemical |
| Sample V-F | 800 ppm of the multipurpose process chemical |

The control and Samples V-A through V-F were stirred for twenty-four (24) hours at ambient temperature (˜71° F.). The performance of the multipurpose process chemical (Samples V-E and V-F) was comparable to that of incumbent product SULFIX™ 9272 (Sample V-B). At the end of the test, Samples V-E and V-F contained no detectable H₂S. The table below summarizes the hydrogen sulfide scavenging observed for each sample:

| | Ratio (H₂S:1 | H₂S at | % H₂S |
|---|---|---|---|
| Sample | part scavenger) | 24 hours (ppm) | reduction |
| Control | — | 3,500 | — |
| Sample V-A | 10 | <10 | >99% |
| Sample V-B | 10 | 1,100 | 69% |
| Sample V-C | 5 | 3,500 | 0% |
| Sample V-D | 2.5 | 3,500 | 0% |
| Sample V-E | 10 | 0 | 100% |
| Sample V-F | 5 | 0 | 100% |

In the foregoing specification, the invention has been described with reference to specific embodiments thereof. However, it will be evident that various modifications and changes can be made thereto without departing from the broader scope of the invention as set forth in the appended claims. Accordingly, the specification is to be regarded in an illustrative rather than a restrictive sense. For example, different imidazoline compounds and cyclic carbonates, treatment procedures, proportions, dosages, temperatures, and amounts not specifically identified or described in this disclosure or not evaluated in a particular Example are still expected to be within the scope of this invention.

The present invention may suitably comprise, consist of, or consist essentially of the elements disclosed and may be practiced in the absence of an element not disclosed. As used herein, the singular forms “a,” “an,” and “the” are intended to include the plural forms as well, unless the context clearly indicates otherwise. As used herein, the term “about” in reference to a given parameter is inclusive of the stated value and has the meaning dictated by the context (e.g., it includes the degree of error associated with measurement of the given parameter). As used herein, the term “and/or” includes any and all combinations of one or more of the associated listed items.

It is claimed:

1. A method for using a multipurpose process chemical comprising the steps of:
reacting an aminoethyl imidazoline with a cyclic carbonate to form the multipurpose process chemical; and
injecting the multipurpose process chemical into a system to address at least one of metal corrosion or hydrogen sulfide accumulation.

2. The method of claim 1, wherein the step of reacting the aminoethyl imidazoline with the cyclic carbonate further comprises deriving the aminoethyl imidazoline from a condensation reaction of a fatty acid and a polyethylene amine.

3. The method of claim 1, wherein the step of reacting the aminoethyl imidazoline with the cyclic carbonate further comprises selecting the cyclic carbonate from a group consisting of propylene carbonate, ethylene carbonate, glycerol carbonate, vinyl carbonate, and combinations thereof.

4. The method of claim 1, wherein the step of reacting the aminoethyl imidazoline with the cyclic carbonate further comprises combining the aminoethyl imidazoline with the cyclic carbonate in a ratio within a range from approximately 1:0.5 to 1:2 (aminoethyl imidazoline:cyclic carbonate).

5. The method of claim 5, wherein the step of reacting the aminoethyl imidazoline with the cyclic carbonate further comprises combining the aminoethyl imidazoline with the cyclic carbonate in a ratio of approximately 1:0.5 (aminoethyl imidazoline:cyclic carbonate).

6. The method of claim 5, wherein the step of reacting the aminoethyl imidazoline with the cyclic carbonate further comprises combining the aminoethyl imidazoline with the cyclic carbonate in a ratio of approximately 1:1 (aminoethyl imidazoline:cyclic carbonate).

7. The method of claim 1, wherein the step of reacting the aminoethyl imidazoline with the cyclic carbonate further comprises combining the aminoethyl imidazoline with an amount of the cyclic carbonate that exceeds what is required to react all of the aminoethyl imidazoline.

8. The method of claim 1, wherein the step of reacting the aminoethyl imidazoline with the cyclic carbonate is conducted at a temperature between about 50° C. and about 120° C.

9. The method of claim 1, wherein the step of injecting the multipurpose process chemical into a system further comprises the step of mixing the multipurpose process chemical into a target fluid within the system, wherein the target fluid contains hydrogen sulfide.

10. The method of claim 9, wherein the step of mixing the multipurpose process chemical into the target fluid comprises adding the multipurpose process chemical in a concentration between about 1 ppm to about 800 ppm of the multipurpose process chemical in the target fluid.

11. A multipurpose process chemical comprising:
a first adduct (i) of the structure: 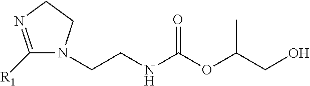
where R₁ is a C₈-C₂₂ alkenyl; and a second adduct (ii) of the structure: 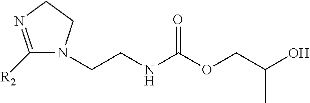
where R₂ is a C₈-C₂₂ alkenyl.

12. The multipurpose process chemical of claim 11, further comprising a compound of the structure: 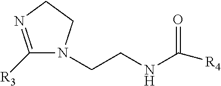
where R₃ is a C₈-C₂₂ alkenyl, and R₄ is an alkyl group derived from a fatty acid.

13. A method for preparing a multipurpose process chemical comprising at least one adduct, wherein the method comprises the steps of:
obtaining an imidazoline synthesized from a fatty acid;
combining the imidazoline with a cyclic carbonate to form a mixture; and
heating the mixture at approximately 80° C. to synthesize the at least one adduct from the imidazoline and the cyclic carbonate.

14. The method of claim 13, wherein the step of obtaining an imidazoline further comprises deriving the imidazoline from a condensation reaction of tall oil fatty acid and diethylenetriamine (DETA).

15. The method of claim 13, wherein the step of combining the imidazoline with the cyclic carbonate further comprises mixing the imidazoline with the cyclic carbonate in a ratio within a range from approximately 1:0.5 to 1:2 (imidazoline:cyclic carbonate).

16. The method of claim 13, wherein the step of combining the imidazoline with the cyclic carbonate further comprises selecting the cyclic carbonate from a group consisting of propylene carbonate, ethylene carbonate, glycerol carbonate, vinyl carbonate, and combinations thereof.

17. The method of claim 13, wherein the step of combining the imidazoline with the cyclic carbonate further comprises mixing the imidazoline with propylene carbonate.

18. The method of claim 17, wherein the at least one adduct comprises a first adduct (i) of the structure: 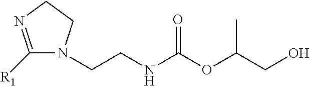
where R₁ is a C₈-C₂₂ alkenyl.

19. The method of claim 18, wherein the at least one adduct further comprises a second adduct (ii) of the structure: 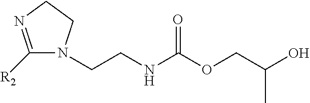
where R₂ is a C₈-C₂₂ alkenyl.

20. The method of claim 13, further comprising the step of:
heating the imidazoline to approximately 80° C. before contacting the imidazoline with the cyclic carbonate to form the mixture.
